(19) United States
(12) Patent Application Publication
(10) Pub. No.: US 2015/0150488 A1
(43) Pub. Date: Jun. 4, 2015

(54) STRESS EVALUATION APPARATUS

(71) Applicants: DENSO CORPORATION, Kariya-city (JP); A SCHOOL CORPORATION KANSAI UNIVERSITY, Osaka (JP)

(72) Inventors: Takahiro ISHIKAWA, Nagoya-city (JP); Kentaro KOTANI, Suita-city (JP); Satoshi SUZUKI, Suita-city (JP); Takafumi ASAO, Suita-city (JP); Susumu ENOKIDA, Suita-city (JP)

(21) Appl. No.: 14/543,943

(22) Filed: Nov. 18, 2014

(30) Foreign Application Priority Data
Nov. 29, 2013 (JP) 2013-247885

(51) Int. Cl. A61B 5/103

(52) U.S. Cl. CPC A61B 5/1036

(57) ABSTRACT

In an evaluation process, a first muscle strength (i.e., muscle potential) of a first muscle responsible for a dorsiflexing action of the ankle joint of a leg manipulating an accelerator pedal is acquired using a first muscle potential sensor. A second muscle strength (i.e., muscle potential) of a second muscle responsible for a plantarflexing action of the ankle joint of the leg manipulating the accelerator pedal is acquired using a second muscle potential sensor. Then, on the basis of time-series variation of each of the muscle potentials acquired using the first and second muscle potential sensors, a synchronous activity level is calculated as an evaluation index. When the difference between the evaluation index and an evaluation standard pre-determined is not less than a prescribed value, it is evaluated that an occupant of a vehicle feels stress as a result of manipulating an in-vehicle information apparatus while driving the vehicle.

### CROSS REFERENCE TO RELATED APPLICATION

The present application is based on Japanese Patent Application No. 2013-247885 filed on Nov. 29, 2013, the disclosure of which is incorporated herein by reference.

### TECHNICAL FIELD

The present disclosure relates to a stress evaluation apparatus that evaluates a stress received by an occupant of a moving body.

### BACKGROUND ART

Patent Literature 1: JP 2005-087486 A (JP 4433739 B2, US 2005-090757 A1)

Conventionally, a stress evaluation apparatus has been known which is mounted in an automobile to evaluate the stress felt by a driver depending on automobile ride quality or as a result of steering (see Patent Literature 1). The stress evaluation apparatus described in Patent Literature 1 measures the contracted state of the masseter muscle of the automobile driver, and consequently evaluates that, as the contraction of the masseter muscle is larger, the stress felt by the driver is higher.

The reason for thus evaluating the stress using the masseter muscle is as follows. When an external force is applied to the head of the driver who is driving the automobile, the masseter muscle contracts so as to hold the position of the head. On the basis of the contracted state of the masseter muscle, the stress resulting from the external force applied to the head can be evaluated.

In an automobile, an in-vehicle information apparatus (e.g., navigation apparatus) is mounted for the purpose of improving the safety and convenience of the automobile. A human-machine interface (HMI) provides an interaction between such an in-vehicle information apparatus and a vehicle occupant and, among such HMIs, complicated HMIs have increased in number.

When a driver who is driving an automobile manipulates an in-vehicle information apparatus via such a complicated HMI, the driver feels stress (mental burden).

In the stress evaluation apparatus described in Patent Literature 1, a consideration has been given to the external force applied to the driver's head as a stress-causing factor (so-called stressor). However, no consideration has been given to a manipulation of in-vehicle information apparatus via an HMI. Thus, the stress evaluation apparatus described in Patent Literature 1 cannot evaluate the stress received by the driver when the driver manipulates an in-vehicle information apparatus via an HMI while driving.

### SUMMARY

It is therefore an object of the present disclosure to evaluate the stress received by an occupant of a moving body or vehicle when the occupant, who is driving the moving body, manipulates an in-vehicle information apparatus via an HMI.

The present disclosure relates to a stress evaluation apparatus which is mounted in a moving body to evaluate the stress received by an occupant of the moving body. In general, in the action (e.g., a manipulation of an accelerator pedal) of an occupant when driving a moving body or vehicle, either one of first and second antagonistic muscles contracts. As a result of conducting study, the present inventors have found that, when an occupant feels stress as a result of manipulating an in-vehicle information apparatus while driving the moving body, both of the first and second antagonistic muscles simultaneously act.

Note that the first muscle mentioned herein is a muscle responsible for the dorsiflexing action of an ankle joint among the muscles of the occupant. Also, the second muscle mentioned herein is a muscle responsible for the plantarflexing action of an ankle joint among the muscles of the occupant. The in-vehicle information apparatus mentioned herein is to be manipulated by the occupant using the upper limb of the occupant via an HMI.

To achieve the above object, according to an example of the present disclosure, a stress evaluation apparatus mounted in a moving body is provided to evaluate stress received by an occupant of the moving body. The stress evaluation apparatus includes a first acquisition section, a second acquisition section, a strength information generation section, and an evaluation section. The first acquisition section acquires a first muscle strength showing a contracted state of a first muscle responsible for a dorsiflexing action of an ankle joint among muscles of the occupant. The second acquisition section acquires a second muscle strength showing a contracted state of a second muscle responsible for a plantarflexing action of the ankle joint among the muscles of the occupant. The strength information generation section generates strength information by associating the first muscle strength acquired by the first acquisition section with the second muscle strength acquired by the second acquisition section. The evaluation section evaluates, based on the strength information generated by the strength information generation section, the stress received by the occupant as a result of the occupant's manipulating an in-vehicle information apparatus with an upper limb of the occupant.

Such a stress evaluation apparatus evaluates the amount of the activity of both of the first and second antagonistic muscles which are simultaneously acting. This allows evaluation of the stress felt by the driver (occupant of the moving body or vehicle) who is driving as a result of manipulating the in-vehicle information apparatus via the HMI. That is, the stress evaluation apparatus of the example of the present disclosure allows objective evaluation of the stress resulting from a manipulation of the in-vehicle information apparatus.

Note that an evaluation section in the present disclosure may perform alternative determination of whether or not stress is received (felt) as evaluation or may also evaluate a quantitative amount of stress.

In particular, in the latter case, in a vehicle control apparatus, the content of moving-vehicle driving assist control can be changed to the safer one in accordance with the amount of stress. This can realize safe driving of the moving vehicle.

According to another example of the present disclosure, a program product stored in a non-transitory computer-readable medium is provided to include instructions for execution by a computer in a moving body to evaluate a stress received by an occupant in the moving body. The instructions permits the computer to achieve the sections included in the stress evaluation apparatus of the above example.

### BRIEF DESCRIPTION OF THE DRAWINGS

The above and other objects, features, and advantages of the present disclosure will become more apparent from the following detailed description made with reference to the accompanying drawings. In the drawings:

FIG. 1 is a block diagram showing a schematic configuration of a stress evaluation system according to an embodiment of the present disclosure;

FIG. 2A is an illustrative view illustrating a muscle (anterior tibial muscle) from which a muscle potential is measured using a first muscle potential sensor;

FIG. 2B is an illustrative view illustrating a muscle (gastrocnemius muscle) from which a muscle potential is measured using a second muscle potential sensor;

FIG. 3 is a block diagram showing a schematic configuration of an in-vehicle information apparatus;

FIG. 4 is a flowchart diagram showing the procedure of an evaluation process;

FIG. 5 is a view showing the result of a demonstration experiment in an embodiment; and

FIG. 6 is a view showing the result of a demonstration experiment in a modification.

### DETAILED DESCRIPTION

An embodiment of the present disclosure will be described below with reference to the drawings.

<Configuration of Stress Evaluation System>

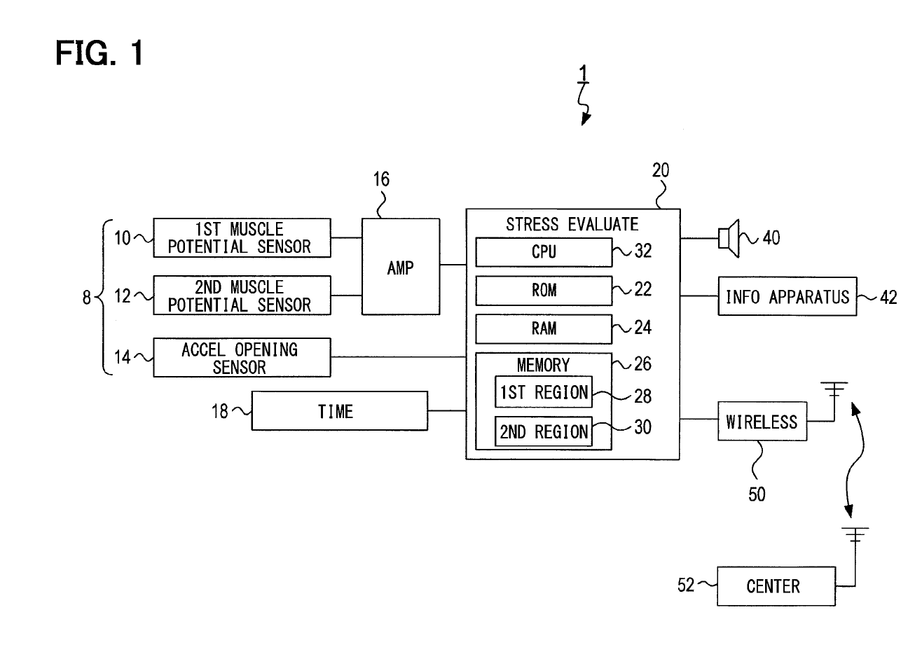

A stress evaluation system 1 shown in FIG. 1 is mounted in an automobile (i.e., vehicle or moving body) to evaluate the stress felt or received by the driver of the automobile.

The stress evaluation system 1 includes a sensor group 8, an amplifier 16, a timer 18, and a stress evaluation apparatus 20.

Among them, the sensor group 8 includes a first muscle potential sensor 10, a second muscle potential sensor 12, and an accelerator opening sensor 14.

The first muscle potential sensor 10 is a known sensor which includes an active electrode and measures a muscle potential using the active electrode. The first muscle potential sensor 10 is attached to the first muscle of a leg manipulating an acceleration pedal among the muscles of the driver and measures the amount of activity of the first muscle as a muscle potential.

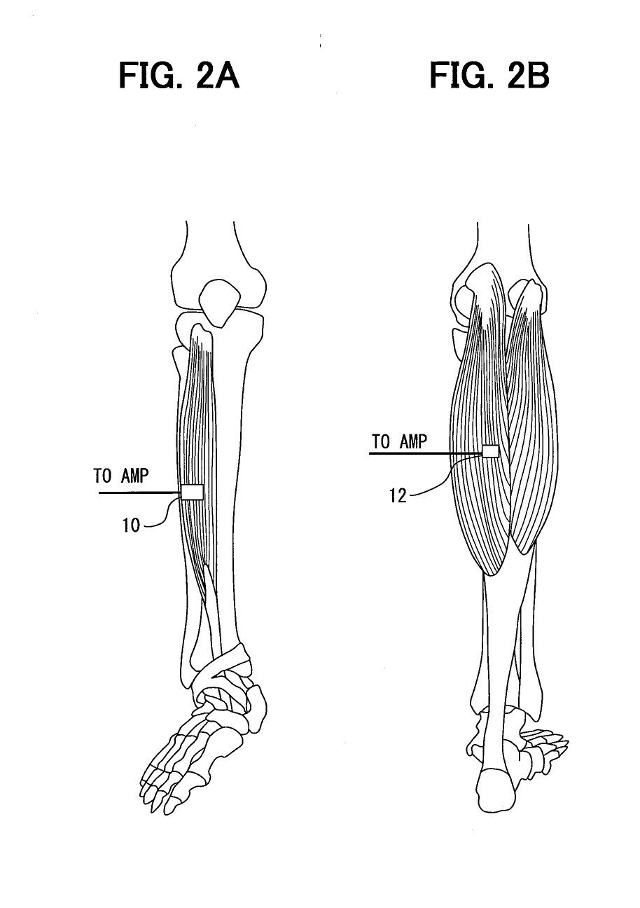

Note that the first muscle mentioned herein is a muscle responsible for the dorsiflexing action of an ankle joint, e.g., at least one of the anterior tibial muscle and peroneus tertius muscle each shown in FIG. 2A. The amount of activity of the first muscle mentioned herein is an example of a first muscle strength, which is information including the contracted state of the first muscle.

The second muscle potential sensor 12 is a known sensor which includes an active electrode and measures a muscle potential using the active electrode. The second muscle potential sensor 12 is attached to the second muscle of the leg manipulating the acceleration pedal among the muscles of the driver and measures the amount of activity of the second muscle as a muscle potential.

Note that the second muscle mentioned herein is the muscle responsible for the plantarflexing action of an ankle joint and is, e.g., at least one of the gastrocnemius muscle, soleus muscle, posterior tibial muscle, long fibular muscle, and short fibular muscle each shown in FIG. 2B. The amount of activity of the second muscle mentioned herein is an example of a second muscle strength, which is information including the contracted state of the second muscle.

The first muscle potential sensor 10 and the second muscle potential sensor 12 in the present embodiment is preferably configured to be wearable such that the two muscle potential sensors 10 and 12 are collectively and simultaneously attachable. As a method for providing such a wearable configuration, it can be considered to incorporate the two muscle potential sensors 10 and 12 into a band to be attached to a leg of the driver.

The accelerator opening sensor 14 is a known sensor which detects an amount of manipulation of (amount of stepping on) an acceleration pedal. The amount of manipulation of the accelerator pedal detected by the accelerator opening sensor 14 will be hereinafter referred to also as an accelerator opening.

The amplifier 16 is a known amplifier which amplifies an input signal and outputs the amplified input signal. The amplifier 16 amplifies signals from the first and second muscle potential sensors 10 and 12. Note that the inputting of the signals from the first and second muscle potential sensors 10 and 12 to the amplifier 16 and the inputting of a signal from the amplifier 16 to the stress evaluation apparatus 20 may be performed either wiredly via a wired communication line or wirelessly.

The time measurement apparatus 18 is a known apparatus which measures a time.

<Configuration of Connecter>

To the stress evaluation system 1, a sound output apparatus 40, an in-vehicle information apparatus 42, and a wireless communication apparatus 50 are connected.

The sound output apparatus 40 outputs a voice in response to a control command from the stress evaluation apparatus 20.

The in-vehicle information apparatus 42 is a known apparatus including a navigation function which provides a route to a destination. The in-vehicle information apparatus 42 in the present embodiment is placed to be embedded in the upper surface or outer surface of the dashboard of the automobile so as to be manipulable by the driver with the upper limb of the driver.

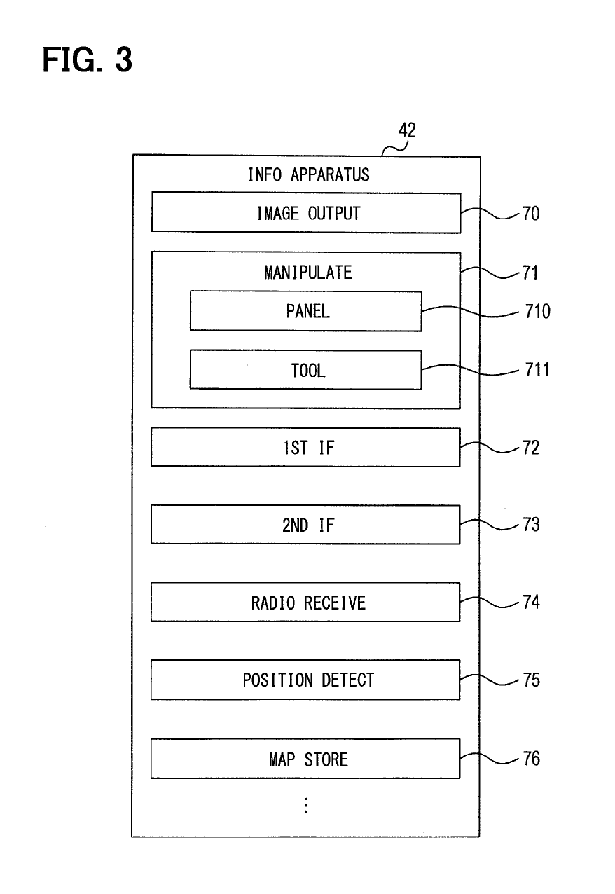

As shown in FIG. 3, the in-vehicle information apparatus 42 includes an image output unit 70, a manipulation unit 71, a first interface 72, a second interface 73, a radio receiver 74, a position detection unit 75, and a map storage unit 76.

Among them, the image output unit 70 is a liquid crystal display which displays various images or the like.

The manipulation unit 71 is a mechanism which receives input information and has a manipulation panel 710 and a manipulation tool 711. The manipulation panel 710 is a known touch panel formed integrally with the image output unit 70. The manipulation tool 711 includes buttons placed around the image output unit 70.

The first interface 72 performs communication between the stress evaluation apparatus 20 and an in-vehicle control apparatus. The in-vehicle control apparatus mentioned herein controls an in-vehicle apparatus (such as, e.g., a cruise control system or a car air conditioner) mounted in an automobile and different from the stress evaluation apparatus 20.

The second interface 73 performs communication with a mobile terminal. The radio receiver 74 receives a radio wave from a radio broadcasting station.

The position detection unit 75 is a known unit which detects the current location of a host vehicle and the azimuth in the direction of travel thereof. To the position detection unit 75, at least a GPS receiver which receives a signal from a GPS satellite, a gyro sensor, and a geomagnetism sensor are connected. The position detection unit 75 detects the current location and the azimuth in the direction of travel in response to signals from the GPS receiver, the gyro sensor, and the geomagnetism sensor.

The map storage unit 76 is a rewritable nonvolatile storage unit which is formed of, e.g., a hard disc drive or a flash memory. In the map storage unit 76, at least map data is stored. The map data includes various data such as node data, link data, cost data, road data, geography data items, mark data, intersection data, facility data, guidance voice data, and voice recognition data.

Such an in-vehicle information apparatus 42 includes, as some of the functions forming the navigation function, a phone-number-based destination retrieval function and a map scroll function. The in-vehicle information apparatus 42 further includes, as a function different from the navigation function, a radio-wave select function, a car-air-conditioner control function, and an audio function.

The phone-number-based destination retrieval function mentioned herein is a known function which displays numeric keypad buttons on the manipulation panel 710 and retrieves a destination on the basis of the phone number input thereto via the displayed numeric keypad buttons. Note that the information related to the input phone number is used not only as information for retrieving a destination during the retrieval of a route, but also as information for specifying a phone number to be called when a phone call is made via the in-vehicle information apparatus 42 or a mobile phone connected to the in-vehicle information apparatus 42.

The map scroll function is a known function which scrolls, when a part of the map image stored in the map storage unit 76 is displayed on the image output unit 70 so as to provide a route to the destination, the map image in response to the manipulation performed via the manipulation unit 71.

The radio-wave select function selects the frequency of a radio wave to be received in response to the manipulation received via the manipulation unit 71. The radio wave at the frequency selected by the function is received and the voice corresponding to the received radio wave is output via the sound output apparatus 40.

The wireless communication apparatus 50 is an apparatus which performs wireless communication with a data center 52. The data center 52 is configured to include mainly a known computer having at least a ROM, a RAM, a CPU, and a storage unit. The data center 52 further includes a wireless communication apparatus which performs wireless communication with the wireless communication apparatus 50. In the data center 52, an evaluation standard is stored, which will be described later in detail.

<Stress Evaluation Apparatus>

The stress evaluation apparatus 20 is an electronic control apparatus configured to mainly include a known computer having at least a ROM 22, a RAM 24, a memory 26, and a CPU 32.

Among them, the ROM 22 stores therein a processing program and data having data contents which need to be held even when a power source is disconnected. The RAM 24 temporarily stores therein a processing program and data. The CPU 32 performs various processes in accordance with the processing programs stored in the ROM 22 and the RAM 24.

The ROM 22 stores therein the processing program for the stress evaluation apparatus 20 to perform an evaluation process of evaluating the stress felt by the driver as a result of manipulating the in-vehicle information apparatus 42. The evaluation process is performed on the basis of the muscle potential from the first muscle potential sensor 10 and the muscle potential from the second muscle potential sensor 12.

The memory 26 is a storage unit which stores therein at least an evaluation index and an evaluation standard to be referenced by the stress evaluation apparatus 20 performing the evaluation process. The memory 26 may also be a nonvolatile storage unit (e.g., a hard disc drive or flash memory) having data contents which are rewritable and need to be held even when a power supply is interrupted (i.e., when the power source is turned OFF).

The memory 26 includes a first storage unit region 28 for storing the evaluation index and a second storage unit region 30 for storing the evaluation standard.

The evaluation index mentioned herein is for evaluating the stress felt by the driver as a result of manipulating the in-vehicle information apparatus 42. The evaluation index results from numerical processing of the muscle potentials measured by the first and second muscle potential sensors 10 and 12. Note that the numerical processing mentioned herein includes the derivation of, e.g., a mean value of the root mean squares (RMSs) of the muscle potentials from the first and second muscle potential sensors 10 and 12 which sequentially change along a time axis and the standard deviation of the RMSs.

The evaluation standard is defined as the evaluation index in a state where the driver feels stress as a result of manipulating the in-vehicle information apparatus. The evaluation standard may be a fixed value calculated in advance by an experiment or may be updated in succession on the basis of the evaluation index derived in the stress evaluation system 1 mounted in another automobile.

In the latter case, the evaluation standard is preferably derived and updated at the data center 52. In this case, the stress evaluation apparatus 20 may also receive the evaluation standard from the data center 52 via the wireless communication apparatus 50 at prescribed time intervals and update the evaluation standard stored in the second storage unit region 30.

<Evaluation Process>

Next, a description will be given of the evaluation process performed by the stress evaluation apparatus 20.

It is noted that a flowchart or the processing of the flowchart in the present application includes sections (also referred to as steps), each of which is represented, for instance, as S110. Further, each section can be divided into several sub-sections while several sections can be combined into a single section. Furthermore, each of thus configured sections can be also referred to as a device or means. Each or any combination of sections explained in the above can be achieved as (i) a software section in combination with a hardware unit (e.g., computer) or (ii) a hardware section, including or not including a function of a related apparatus; furthermore, the hardware section may be constructed inside of a microcomputer.

The evaluation process is activated when an ignition switch is turned ON. When activated, the evaluation process is repeatedly performed during the period before the ignition switch is turned OFF.

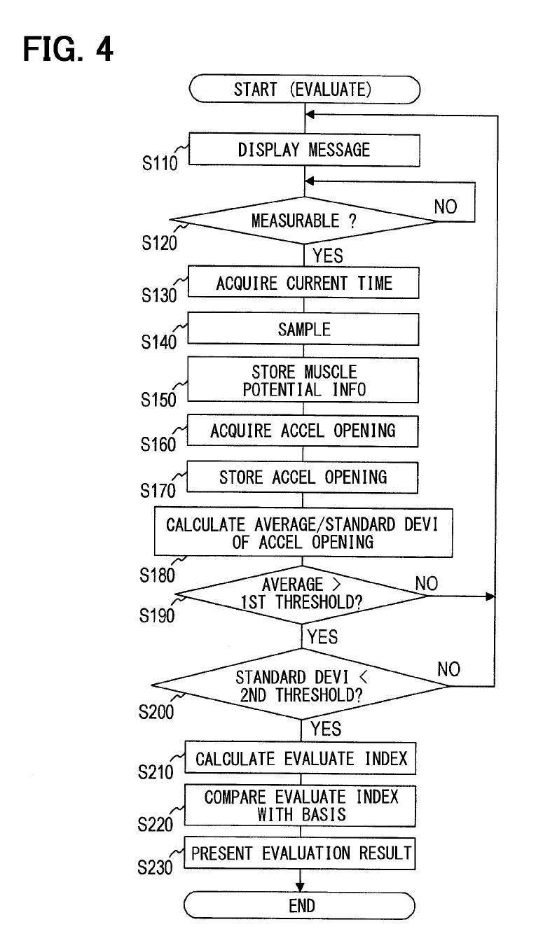

When activated, as shown in FIG. 4, the evaluation process first displays a message on the image output unit 70 of the in-vehicle information apparatus 42 (S110). The message displayed on the image output unit 70 prompts the driver to attach the first and second muscle potential sensors 10 and 12 respectively to the first and second muscles.

Subsequently, it is determined whether or not the measurement of the muscle potentials by the first and second muscle potential sensors 10 and 12 is possible (S120). In S120, it is determined that the measurement of the muscle potentials by the individual muscle potential sensors 10 and 12 is possible as long as the first and second muscle potential sensors 10 and 12 have been attached respectively to the first and second muscles.

As a result of the determination at S120, when the measurement of the muscle potentials by the first and second muscle potential sensors 10 and 12 is impossible (NO at S120), the stress evaluation apparatus 20 stands by until the measurement becomes possible. Note that the stress evaluation apparatus 20 in a stand-by mode may also prompt the driver to correctly reattach the first and second muscle potential sensors 10 and 12.

When the measurement of the muscle potentials by the first and second muscle potential sensors 10 and 12 becomes possible (YES at S120), a current time is acquired from the timer 18 (S130).

Subsequently, the measurement results (i.e., muscle potentials) from the first and second muscle potential sensors 10 and 12 are sampled in sampling periods determined in advance (S140). That is, at S140, the respective muscle potentials measured by the first and second muscle potential sensors 10 and 12 are acquired.

Then, muscle potential information is generated by associating the sampling results (acquired muscle potentials) with the current time acquired at S130 and stored in the RAM 24 (S150).

Then, from the accelerator opening sensor 14, the accelerator opening (an example of manipulation amount information) is acquired (S160). The acquired acceleration opening is associated with the current time acquired at S130 and stored in the RAM 24 (S170).

Subsequently, from among the accelerator openings stored in the RAM 24, all the accelerator openings which satisfy a prescribed condition are acquired. The mean value (hereinafter referred to as “opening mean value”) of the acquired accelerator openings and the standard deviation (hereinafter referred to as “opening standard deviation”) of the accelerator openings are calculated (S180). The prescribed condition mentioned herein is that the time associated with the accelerator opening is included in a prescribed time period starting subsequently from the current time.

Note that the opening mean value is obtained by subjecting the accelerator openings each satisfying the prescribed condition to known arithmetic averaging. The opening standard deviation is obtained as a known standard deviation using the acceleration openings each satisfying the prescribed condition as a known sample set.

Then, it is determined whether or not the calculated opening mean value is larger than a first threshold determined in advance (S190). The first threshold has been determined in advance as the accelerator opening indicating that the accelerator pedal has been stepped on so as to permit the automobile to run at a speed not less than a prescribed speed.

As a result of the determination at S190, when the opening mean value is less than the first threshold value (NO at S190), the process returns to S110. Conversely, as a result of the determination at S190, when the opening mean value is larger than the first threshold value (YES at S190), it is determined whether or not the opening standard deviation is smaller than a second threshold value (S200). The second threshold value has been determined in advance as variations in accelerator opening corresponding to the fluctuations in the vehicle speed of the automobile within the prescribed range determined in advance.

As a result of the determination at S200, when the opening standard deviation is not less than the second threshold value (NO at S200), the process returns to S110. Suppose the case when the vehicle speed of the automobile is less than the prescribed speed or the case when fluctuations in the vehicle speed of the automobile are large even though the vehicle speed is not less than the prescribed speed. In either case, it is highly possible that, in the first muscle or the second muscle, contraction resulting from a manipulation of the accelerator pedal by the driver is observed. Accordingly, the process returns to S110 without evaluating the stress felt by the driver.

Conversely, as a result of the determination at S200, when the opening standard deviation is smaller than the second threshold value, the process advances to S210. That is, when the vehicle speed of the automobile is not less than the prescribed speed and fluctuations in vehicle speed are within the prescribed range, it is highly possible that, in the muscle potential in the first or second muscle, contraction resulting from a manipulation of the in-vehicle information apparatus 42, rather than from a manipulation of the accelerator pedal, is observed. Accordingly, the process moves to S210 where the stress felt by the driver is evaluated.

In S210, as the evaluation index (an example of strength information), a synchronous activity level M is derived. The derivation of the evaluation index may be performed appropriately in accordance with the following procedure.

First, from among all the muscle potential information stored in the RAM 24, a signal (hereinafter referred to as “first-muscle time-series data”) from the first muscle potential sensor 10 which is along a time axis and satisfies the prescribed condition and a signal (hereinafter referred to as “second-muscle time-series data”) from the second muscle potential sensor 12 which is along the time axis and satisfies the prescribed condition are subjected to pre-processing.

In the pre-processing, first, the first-muscle time-series data and the second-muscle time-series data are caused to pass through a low-pass filter which passes a signal at a frequency not more than a prescribed frequency (e.g., 200 [Hz]) and a high-pass filter which passes a signal at a frequency not less than a set frequency (e.g., 15 [Hz]). In the pre-processing, each of the first-muscle time-series data and the second-muscle time-series data that have passed through the low-pass filter and the high-pass filter is subjected to full-wave rectification.

Then, in the present embodiment, in accordance with the following formula (1), the synchronous activation level M is calculated.

$$\begin{matrix}[ Formula 1 ] & \\ M = \sqrt{\sqrt{\frac{1}{T} ( {\sum }_{t = 0}^{T} {EMGzen ( t )}^{2} )} \times \sqrt{\frac{1}{T} ( {\sum }_{t = 0}^{T} {EMGhih ( t )}^{2} )}} & ( 1 )\end{matrix}$$

In the formula (1), EMGzen(t) is the first-muscle time-series data subjected to the pre-processing and EMGhih(t) is the second-muscle time-series data subjected to the pre-processing. The mark T in the formula (1) is an interval in moving averaging (the number of points used to calculate an average by moving averaging).

That is, in the derivation of the evaluation index, the square value of each of the first-muscle time-series data EMGzen(t) and the second-muscle time-series data EMGhih(t) (each of the sampled points of the muscle potentials) that have been subjected to the pre-processing is calculated. Then, each of the square value of the first-muscle time-series data EMGzen(t) and the square value of the second-muscle time-series data EMGhih(t) is subjected to simple moving averaging on the basis of each prescribed number of points T (e.g., on a per 100-point basis). In addition, the respective square roots of the results of the simple moving averaging are calculated and the square root of the product of the squared roots is calculated as the synchronous activity level M (i.e., evaluation index).

In other words, at S210, the stress felt by the driver as a result of manipulating the in-vehicle information apparatus 42 is calculated as the objective numerical value of the synchronous activity level M.

Subsequently, in the evaluation process, the synchronous activity level M derived at S210 is compared to the evaluation standard stored in the memory 26 (S220). Specifically, at S220, the difference between the synchronous activity level M and the evaluation standard is derived. It is determined by evaluation that, as the difference is larger, the received stress is higher. That is, at S220, it is determined by evaluation that, when the difference between the evaluation index and the evaluation standard is not less than the prescribed value, the driver feels stress as a result of manipulating the in-vehicle information apparatus 42.

Note that the evaluation result in the present embodiment is expressed by a stress level. Depending on the difference between the synchronous activity level M and the evaluation standard, three stress levels are set. Stress Level 1 signifies that the received stress is substantially zero. Stress Level 2 signifies that the received stress is at a level at which the driver needs to be attentive while driving an automobile. Stress Level 3 signifies that the received stress is at a level at which driving an automobile is dangerous (equivalent to inhibition of manipulations).

In the evaluation process, when the received stress is evaluated to be at Stress level 2 or Stress level 3 as a result of the evaluation at S220, a user presentation process is performed (S230).

In the user presentation process, when the evaluation result is, e.g., Stress Level 3, manipulations of the in-vehicle information apparatus 42 are totally inhibited and the notification thereof is output from the sound output apparatus 40. On the other hand, when the evaluation result is Stress Level 2, those of manipulations of the in-vehicle information apparatus 42 which give high stress when performed during driving (hereinafter referred to as “high-stress manipulations”) are inhibited and the notification thereof is output from the sound output apparatus 40.

The high-stress manipulations mentioned herein may include a manipulation in which, e.g., the in-vehicle information apparatus 42 receives inputs of a variety of information via the manipulation unit 71 to perform the destination retrieval function using a phone number in the in-vehicle information apparatus 42. The high-stress manipulations mentioned herein may also include a manipulation in which, e.g., the in-vehicle information apparatus 42 receives inputs of a variety of information via the manipulation unit 71 to perform the radio wave select function in the in-vehicle information apparatus 42.

Note that, when the result of the evaluation at S220 is Stress Level 1, switching may also be performed to enable the manipulations inhibited at S230 to be performed.

Then, the evaluation process is repeatedly performed during the period before the ignition switch is turned OFF and ended when the ignition switch is turned OFF.

That is, in the evaluation process, a first muscle strength (i.e., muscle potential) showing the contracted state of the first muscle responsible for the dorsiflexing action of the ankle joint of a leg manipulating the accelerator pedal is acquired by measurement using the first muscle potential sensor 10. Also in the evaluation process, a second muscle strength (i.e., muscle potential) showing the contracted state of the second muscle responsible for the plantarflexing action of the ankle joint of the leg manipulating the accelerator pedal is acquired by measurement using the second muscle potential sensor 12.

Further in the evaluation process, on the basis of the first-muscle time-series data and the second-muscle time-series data, the synchronous activity level M is calculated as the evaluation index. Then, it is determined by evaluation that, when the difference between the evaluation index and the evaluation standard is not less than a prescribed value, it is determined by evaluation that the vehicle occupant feels stress as a result of manipulating the in-vehicle information apparatus 42 while driving an automobile.

<Demonstration Experiment>

Next, a description will be given of the demonstration experiment performed by the present inventors to demonstrate the mechanism of the present disclosure and the result of the demonstration experiment.

First, in the demonstration experiment, an experiment having a content determined in advance was performed on a plurality of subjects under test (drivers). The content of the demonstration test is simultaneous execution of two tasks by the driver of an automobile. The two tasks include a main task related to the driving of the automobile and subtasks related to manipulations of the in-vehicle information apparatus 42.

The main task is to cause the driver to drive the host vehicle in such a manner as to follow a vehicle driving ahead at a given speed, while keeping an inter-vehicular distance kept by the driver when normally driving the vehicle.

On the other hand, the sub-tasks include a no manipulation task (CONTROL), a hand movement task (HAND), a map scroll task (MAP), a radio-wave select task (RADIO), and a phone-number-based destination retrieval task (TEL).

The no manipulation task (CONTROL) is not to manipulate the in-vehicle information apparatus 42 at all. The hand movement task (HAND) is to move one of the hands to a specified region determined in the in-vehicle information apparatus 42.

The map scroll task (MAP) is to scroll the map displayed on the image output unit 70 in accordance with the content of the instruction displayed on the image output unit 70. The radio-wave select task (RADIO) is to select the frequency of a radio wave to be received in accordance with the content of the instruction displayed on the image output unit 70. The phone-number-based destination retrieval task (TEL) is to set the destination corresponding to the phone number input in accordance with the content of the instruction displayed on the image output unit 70.

Note that, in the demonstration experiment, when each of the subjects under test drove the automobile in a test course, the subject under test simultaneously executed the main task and the subtask. During the simultaneous execution of the main task and the subtask, the muscle potential in the anterior tibial muscle of a leg manipulating the accelerator pedal was continuously measured using the first muscle potential sensor 10, while the muscle potential in the gastrocnemius muscle of the leg manipulating the accelerator petal was continuously measured using the second muscle potential sensor 12. Then, on the basis of the result of analyzing the measured muscle potentials in the anterior tibial muscle and the gastrocnemius muscle, the relationship between the stress resulting from each of the manipulations of the in-vehicle information apparatus 42 and the synchronous activity level M was evaluated.

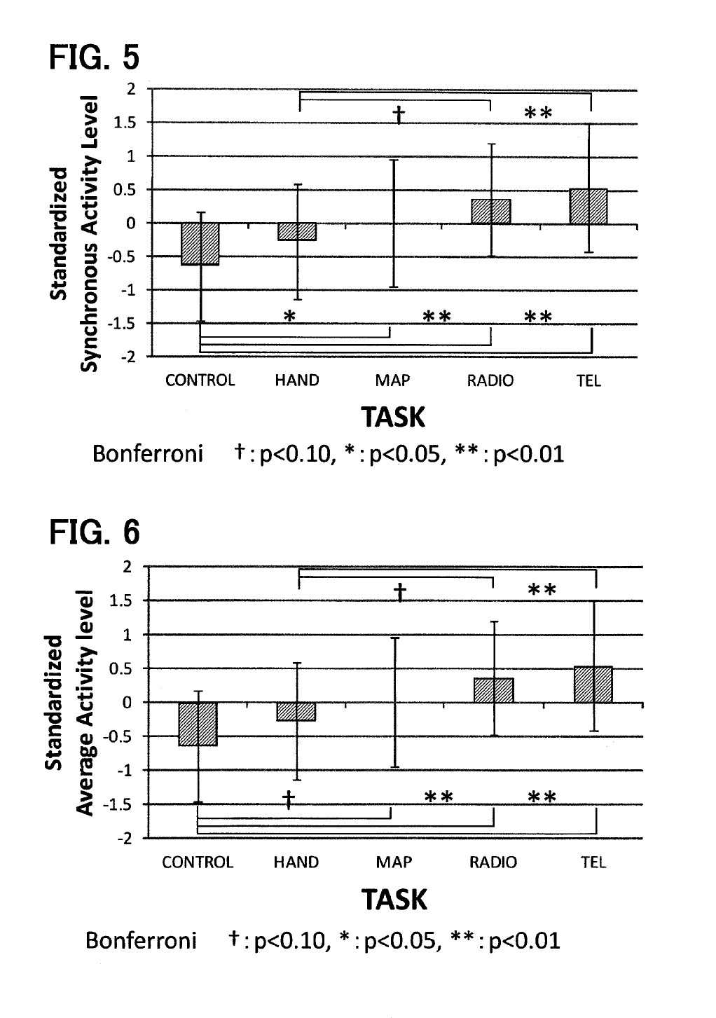

The results of the evaluation are shown in FIG. 5. FIG. 5 shows the relationship between each of the subtasks (TASK) and the synchronous activity level M (Standardized Synchronous Activity level in the drawing). However, the synchronous activity level M shown herein is a mean value and has been standardized among the subtasks for each of the subjects under test.

The relationship shown in FIG. 5 is obtained as a result of a one-way analysis of variance with respect to subtask-factor and multiple comparison based on the Bonferroni method. Note that the cross mark “†” in FIG. 5 indicates significant difference at 10%. The mark “**” indicates significant difference at 1%.

As shown in FIG. 5, it can be said that the synchronous activity levels M when the radio-wave select task and the phone-number-based destination retrieval task were performed are significantly higher than the synchronous activity level when the no manipulation task (i.e., only the main task) was performed.

Accordingly, the present inventors have been able to find that the synchronous activity level M obtained from time-series variation of the anterior tibial muscle of the leg manipulating the accelerator pedal and time-series variation of the gastrocnemius muscle of the leg manipulating the accelerator pedal shows a reaction to the stress resulting from a manipulation of the in-vehicle information apparatus 42, i.e., mental burden.

In addition, it can be seen that the synchronous activity level M is higher as the difficulty level of a subtask is higher and the driver receives higher stress as the difficulty level of the subtask is higher. Note that the difficulty level of the subtask is higher as the number of times the in-vehicle information apparatus 42 is manipulated via the HMI is larger. That is, the phone-number-based destination retrieval task (TEL), the radio-frequency select task (RADIO), the map scroll task (MAP), the hand movement task (HAND), and the no manipulation task (CONTROL) have progressively lower difficulty levels in this order.

### Effect of Embodiment

As described above, the present inventors have found that the stress resulting from a manipulation of the in-vehicle information apparatus 42 causes a change in the synchronous activity level M obtained by evaluating the amount of the activity of the both first and second antagonistic muscles which are simultaneously acting.

On the basis of the finding, the stress evaluation apparatus 20 evaluates the stress received by the driver in accordance with the synchronous activity level M. Thus, with the stress evaluation apparatus 20, it is possible to evaluate the stress received by a driver (vehicle occupant) who is driving as a result of manipulating the in-vehicle information apparatus 42 via an HMI.

In other words, the stress evaluation apparatus 20 allows objective evaluation of the stress resulting from a manipulation of the in-vehicle information apparatus.

When the driver is manipulating the accelerator pedal so as to accelerate/decelerate the automobile, it is highly possible that only either one of the first and second muscles of the driver contracts. On the other hand, when the driver of the automobile is manipulating the accelerator pedal so as to drive the automobile at a given speed not less than a prescribed speed, it is highly possible that neither of the first and second muscles of the driver largely contracts due only to the manipulation of the accelerator pedal. Each of the first and second muscles of the driver is rather likely to contract when the driver feels stress as a result of manipulating the in-vehicle information apparatus 42.

Accordingly, in the stress evaluation apparatus 20, conditions for performing stress evaluation are assumed to be that the vehicle speed of the automobile is not less than the prescribed speed and fluctuations in vehicle speed are within a prescribed range.

The stress evaluation apparatus 20 which evaluates the stress received by the driver under such conditions allows more precise evaluation of the stress received by the driver.

The stress evaluation apparatus 20 also inhibits the driver from performing the high-stress manipulations among the manipulations performed on the in-vehicle information apparatus 42 depending on the magnitude of the stress received by the driver.

Therefore, with the stress evaluation apparatus 20, it is possible to change the content of moving-vehicle driving assist control to the safer one in accordance with the amount of stress received by the driver and realize safe driving of a moving vehicle.

### Other Embodiments

While the embodiment of the present disclosure has been described heretofore, the present disclosure is not limited to the foregoing embodiment. It will be appreciated that the present disclosure can be practiced in various forms within the scope not departing from the gist thereof.

For example, at S210 in the evaluation process of the foregoing embodiment, the synchronous activity level M has been calculated as the evaluation index. However, the evaluation index to be calculated at S210 is not limited to the synchronous activity level M. That is, the evaluation index in the present disclosure may be an average activity level S or a simultaneous activity rate B.

The average activity level S mentioned herein may be calculated appropriately in accordance with the following formula (2).

$$\begin{matrix}[ Formula 2 ] & \\ S = \sqrt{\frac{1}{T} {\sum }_{t = 0}^{T} ( EMGzen ( t ) \times EMGhih ( t ) )} & ( 2 )\end{matrix}$$

That is, as the average activity level S, the product of the first-muscle time-series data EMGzen(t) and the second-muscle time-series data EMGhih(t) is calculated. Then, the product of the first-muscle time-series data EMGzen(t) and the second-muscle time-series data EMGhih(t) is subjected to simple moving averaging on the basis of each prescribed number of points T (e.g., on a per 100-point basis). The square root of each of the results of the simple moving averaging is calculated as the average activity level (i.e., evaluation index).

FIG. 6 shows the result of an experiment for demonstrating that such an average activity level S shows a reaction to the stress resulting from a manipulation of the in-vehicle information apparatus 42, i.e., mental burden. Since the content of the demonstration experiment is as described above in the foregoing embodiment, a detailed description thereof is omitted here.

FIG. 6 shows the relationship between each of the subtasks (TASK) and the average activity level S (Standardized Average Activity Level in the drawing). However, the average activity level S shown herein is a mean value and has been standardized among the subtasks for each of the subjects under test.

The relationship shown in FIG. 6 is obtained as a result of a one-way analysis of variance with respect to subtask-factor and multiple comparison based on the Bonferroni method. Note that the cross mark “†” in FIG. 6 indicates significant difference at 10%. The mark “**” indicates significant difference at 1%.

As shown in FIG. 6, it can be said that the average activity levels S when the radio-wave select task and the phone-number-based destination retrieval task were performed are significantly higher than the average activity level when the no manipulation task (i.e., only the main task) was performed.

Thus, the average activity level S shows a reaction to the stress resulting from a manipulation of the in-vehicle information apparatus 42, i.e., metal burden.

As a result, even when the average activity level S is used as the evaluation index, the same effect as obtained from the stress evaluation apparatus 20 of the foregoing embodiment can be obtained.

Note that the simultaneous activity rate B as the evaluation index may be calculated appropriately by dividing the synchronous activity level M by the average activity level S. Even when such a simultaneous activity rate B is used as the evaluation index also, the same effect as obtained from the stress evaluation apparatus 20 of the foregoing embodiment can be obtained.

In the foregoing embodiment, the navigation apparatus has been assumed as the in-vehicle information apparatus 42. However, the in-vehicle information apparatus 42 is not limited thereto. For example, the in-vehicle information apparatus 42 may be an audio-dedicated apparatus, a navigation-dedicated apparatus, or a car-air-conditioning-dedicated apparatus. That is, the in-vehicle information apparatus 42 may be any in-vehicle apparatus which performs various processing in accordance with information input thereto via the manipulation unit 71 to be manipulated by the driver with his upper limb to perform a specified function, i.e., any in-vehicle information apparatus to be manipulated by a vehicle occupant with the upper limb of the vehicle occupant.

In the foregoing embodiment, the first and second muscle potential sensors 10 and 12 have been attached respectively to the first and second muscles of a leg manipulating the accelerator pedal to measure the muscle potentials. However, the leg to which the first and second muscle potential sensors 10 and 12 are attached may also be the leg opposite to the leg manipulating the accelerator pedal. That is, the muscle potentials for deriving the evaluation index may also be measured from the leg opposite to the leg manipulating the accelerator pedal.

In the stress evaluation apparatus 20 in the foregoing embodiment, a target person of stress evaluation is assumed to be a driver. However, in the stress evaluation apparatus 20, the target person of stress evaluation is not limited thereto and may also be, e.g., any vehicle occupant.

The stress evaluation system 1 in the foregoing embodiment has been mounted in an automobile. However, an object in which the stress evaluation system 1 is to be mounted is not limited to an automobile. The stress evaluation system 1 may also be mounted in a moving body or vehicle such as, e.g., an electric train, an aircraft, or a ship.

While the present disclosure has been described with reference to preferred embodiments thereof, it is to be understood that the disclosure is not limited to the preferred embodiments and constructions. The present disclosure is intended to cover various modification and equivalent arrangements. In addition, while the various combinations and configurations, which are preferred, other combinations and configurations, including more, less or only a single element, are also within the spirit and scope of the present disclosure.

What is claimed is:

1. A stress evaluation apparatus mounted in a moving body to evaluate stress received by an occupant of the moving body, comprising:
a first acquisition section that acquires a first muscle strength showing a contracted state of a first muscle responsible for a dorsiflexing action of an ankle joint among muscles of the occupant;
a second acquisition section that acquires a second muscle strength showing a contracted state of a second muscle responsible for a plantarflexing action of the ankle joint among the muscles of the occupant;
a strength information generation section that generates strength information by associating the first muscle strength acquired by the first acquisition section with the second muscle strength acquired by the second acquisition section; and
an evaluation section that evaluates, based on the strength information generated by the strength information generation section, the stress received by the occupant as a result of the occupant's manipulating an in-vehicle information apparatus with an upper limb of the occupant.

2. The stress evaluation apparatus according to claim 1, wherein
the evaluation section evaluates the stress based on a result of comparing the strength information generated by the strength information generation section to an evaluation standard, which is defined in advance as basis strength information in a state where the occupant has received stress due to manipulating the in-vehicle information apparatus during driving the moving body.

3. The stress evaluation apparatus according to claim 2, wherein
the evaluation section evaluates that, when a difference between the strength information generated by the strength information generation section and the evaluation standard is not less than a prescribed value determined in advance, the occupant has received stress.

4. The stress evaluation apparatus according to claim 1, wherein
the first muscle is at least one muscle among two muscles that are an anterior tibial muscle and a peroneus tertius muscle.

5. The stress evaluation apparatus according to claim 1, wherein
the second muscle is at least one muscle among four muscles that are a gastrocnemius muscle, a soleus muscle, a posterior tibial muscle, a long fibular muscle, and a short fibular muscle.

6. The stress evaluation apparatus according to claim 1, further comprising:
a manipulation amount acquisition section that acquires manipulation amount information showing an amount of manipulation of an accelerator pedal of the moving body, wherein
the evaluation section performs evaluation of the stress received by the occupant when the amount of manipulation shown by the manipulation amount information acquired by the manipulation amount acquisition section indicates that a speed of the moving body is not less than a prescribed speed determined in advance and a change in the speed is within a prescribed range determined in advance.

7. A program product stored in a non-transitory computer-readable storage media, comprising instructions for execution by a computer in a moving body vehicle to evaluate a stress received by an occupant in the moving body, the instructions comprising:
acquiring a first muscle strength showing a contracted state of a first muscle responsible for a dorsiflexing action of an ankle joint among muscles of the occupant;
acquiring a second muscle strength showing a contracted state of a second muscle responsible for a plantarflexing action of the ankle joint among the muscles of the occupant;
generating strength information by associating the first muscle strength acquired with the second muscle strength acquired; and
evaluating, on the basis of the strength information generated, the stress received by the occupant as a result of the occupant's manipulating an in-vehicle information apparatus with an upper limb of the occupant.
